(19) United States
(12) Patent Application Publication
(10) Pub. No.: US 2008/0113952 A1
(43) Pub. Date: May 15, 2008

(54) Combination Therapy For Treating Heart Disease

(75) Inventor: Ajay Gupta, Cerritos, CA (US)

Correspondence Address: Casimir Jones, S.C., 440 Science Drive, Suite 203, Madison, WI, 53711, US

(73) Assignee: Ajay Gupta, Cerritos, CA (US)

(21) Appl. No.: 11/847,660

(22) Filed: Aug. 30, 2007

Related U.S. Application Data
(63) Continuation of application No. 11/016,674, filed on Dec. 17, 2004, now abandoned.

(51) Int. Cl. A61K 31/58; A61K 31/4164

(52) U.S. Cl. 514/171; 514/396

(57) ABSTRACT

A combination therapy and co-therapy method for administering therapeutic doses of an aldosterone antagonist agent and a metolazone-related compound to a subject in need of treatment for hypertension, congestive heart failure, and chronic kidney disease are provided. A pharmaceutical composition comprising these therapeutic agents is also provided.

### CROSS-REFERENCE TO RELATED APPLICATIONS

None.

### STATEMENT REGARDING FEDERALLY SPONSORED RESEARCH & DEVELOPMENT

No Federally sponsored research & development was used in making this invention.

### FIELD OF THE INVENTION

The present invention relates to a combination therapy and co-therapy method for administering therapeutic doses of an aldosterone antagonist agent and a metolazone-related compound to a subject in need of treatment for hypertension, congestive heart failure, and chronic kidney disease. A pharmaceutical composition is also provided.

### BACKGROUND OF THE INVENTION

The clinical syndrome of heart failure is the penultimate end-point for myriad diseases that affect the heart. Heart failure is one of the most common causes of disability and death in the United States and other industrialized nations. Nearly 5 million Americans have heart failure today, the majority of whom are older adults with serious co-existing conditions, including hypertension, hyperlipidemia, and diabetes mellitus. Heart failure is the reason for at least 20% of all hospital admissions among persons older than 65.

Heart failure is largely preventable, primarily through the control of blood pressure and other vascular risk factors. Numerous randomized controlled trials have demonstrated the health benefits associated with a variety of interventions for prevention and treatment of cardiovascular disease. Since heart failure is a clinical syndrome arising from diverse causes and is accompanied by adverse changes in physiological function of organs other than the heart, the appropriate selection of therapeutic agents to yield improvements in cardiovascular morbidity and survival at the various stages of cardiovascular disease frequently requires the concurrent administration of drugs from several classes of therapeutic agents, including angiotensin-converting-enzyme (ACE) inhibitors, angiotensin-receptor antagonists, beta-blockers, hydroxymethylglutaryl coenzyme A reductase inhibitors (statins), and aldosterone antagonists.

### 1. Cellular Actions of Aldosterone

Aldosterone is a steroid hormone secreted by the adrenal gland. The primary site of pharmacological action of aldosterone is at mineralocorticoid receptors in the epithelium of the distal nephron, colon, and rectum, where it promotes sodium absorption and potassium excretion. Aldosterone receptors also have been located on non-epithelial sites in blood vessels, brain, and heart. [Bonvalet J P, Alfaidy N, Farman N, et al. Euro Heart J 93:S92-S97, Suppl N (1995); Komel L. Am J Hypertens 7:100-103 (1994); Lombes M, Oblin M E, Gasc J M, et al. Circ Res 71:503-510 (1992); Tanaka J, Fujita H, Matsuda S, et al. Glia 20:23-27 (1997)]

Numerous studies over the past 10 years suggest that the non-epithelial actions of mineralocorticoids are responsible for their vascular and myocardial fibrotic and trophic effects. [See, by way of example, Brilla C G, Weber K T. J Lab Clin Med 120:893-901 (1992); Ullian M E, Schelling J R, Linas S L. Hypertension 20:67-73 (1992); Young M, Fullerton M, Dilley R, et al. J Clin Invest 93:2578-2583 (1994)] In addition, sites of aldosterone formation outside the adrenal gland have been discovered, including human endothelial cells and vascular smooth muscle cells (VSMC) [Hatakeyama H, Miyamori L, Fujita T, et al. J Biol Chem 289:24318-24320 (1994) and myocardial cells in animal studies [Silvestre J S, Robert V, Heymes C, et al. J Biol Chem 273:4883-4891 (1988)]. Several studies [including, by way of example, Brilla C G, Weber K T. J Lab Clin Med 120:893-901 (1992); Young M, Fullerton M, Dilley R, et al. J Clin Invest 93:2578-2583 (1994)] have linked mineralocorticoids with myocardial fibrosis through stimulation of collagen formation in myocardial cells.

Circulating aldosterone may mediate vascular fibrosis by the direct interaction of this steroid hormone with high affinity low-capacity corticoid receptors located in the cytosol of vascular fibroblasts. When activated, the receptor loses its heat-shock protein, and its monomeric form reaches the cells nucleus, where it binds to DNA within its binding region to initiate the expression of messenger RNA for type I collagen synthesis (or other proteins involved in collagen synthesis) [Weer K T, Anversa P, Armstrong P W, et al. J Am Coll Cardiol 20:3-16 (1992)].

Ullian et al. [Ullian M E, Schelling J R, Linas S L. Hypertension 20:67-73 (1992)] showed that aldosterone may promote VSMC hypertrophy by inducing upregulation of angiotensin II receptors, thus potentiating the pressor responses of angiotensin II.

Clinically, emphasis has been placed on minimizing hyperaldosteronism as a basis for optimizing treatment of patients having heart disease. The effect of aldosterone on target tissues can be blocked by aldosterone receptor antagonists that include, by way of example, spironolactone and eplerenone. The former is a non-selective aldosterone blocker, whereas the latter is a selective aldosterone blocker.

### 2. Extrarenal Adverse Effects of Aldosterone and the Benefits of Aldosterone Antagonists

2.1 Role of aldosterone in heart failure. Farquiharson and Struthers [Farquiharson C A J, Struthers A D. Circulation 101:594-597 (2000)] indirectly showed that aldosterone could have a role in endothelial dysfunction in chronic heart failure. They performed a randomized, placebo-controlled, double-blind, crossover study of 10 patients with New York Heart Association Classes II and III chronic heart failure on standard diuretic and ACE inhibitor therapy, comparing 50 mg/day of spironolactone for 1 month versus placebo. Forearm vascular endothelial function was assessed by bilateral forearm venous occlusion plethysmography using acetylcholine and N-monomethyl-L-arginine (L-NMMA), with sodium nitroprusside as a control vasodilator. The aldosterone antagonist, spironolactone, substantively increased forearm blood flow response to acetylcholine compared with placebo, with an associated increase in vasoconstriction caused by L-NMMA. They concluded that antagonizing the aldosterone receptor improves endothelial dysfunction and increases nitric oxide bioactivity in chronic heart failure.

In patients with heart failure, circulating levels of aldosterone become elevated in response to stimulation by angiotensin II, and there is a decrease in the hepatic clearance of aldosterone due to hepatic congestion. Aldosterone stimulates the retention of salt, myocardial hypertrophy, and potassium excretion. (For a review, see Jessup N, Brozena S. N Engl J Med 348:20, 2007-2080 (2003).] The Randomized Aldactone Evaluation Study (RALES) showed that when added to a standard treatment (including an angiotensin converting enzyme inhibitor), a low dose of the aldosterone antagonist spironolactone reduces the risk of death by 30% over an average follow-up period of two years in carefully selected patients with current or recent heart failure. [Pitt B, Zannad F, Remme W J, et al. N Engl J Med 341:709-717 (1999).] Among these closely monitored patients, there was a low incidence of serious adverse events, including renal dysfunction and hyperkalemia, in the spironolactone group.

2.2 Role of aldosterone in progressive renal disease. Recent evidence also suggests that aldosterone is an important factor in causing progressive renal disease through both hemodynamic effects and direct cellular actions. A number of experimental models are consistent with the concept that aldosterone may have a pathogenetic role in mediating renal injury. In a recent study reported by Quan et al. [Quan Y Z, Walker M, Hill G S. Kidney Int 1992; 41:326-333 (1992)], for example, hypertension, proteinuria, and structural renal injury were less prevalent in rats that underwent subtotal nephrectomy with adrenalectomy compared with rats that underwent partial nephrectomy but had intact adrenal glands. This occurred despite large doses of replacement glucocorticoid (aldosterone was not replaced) in the adrenalectomized rats.

The role of aldosterone has been dissociated from that of angiotensin II in the progression of renal disease. Greene et al. [Greene E, Kern S, Hostetter T H. J Clin Invest 98:1063-1068 (1996)] evaluated four treatment groups (sham-operated rats, untreated remnant rats, remnant rats treated with losartan and enalapril, and remnant rats treated with losartan and enalapril followed by aldosterone infusion) to distinguish the relative importance of aldosterone in the progression of renal injury. They observed that remnant rats had a 10-fold elevation in aldosterone levels in comparison with sham-operated rats. Conversely, remnant rats undergoing treatment with losartan and enalapril manifested suppressed aldosterone levels and a decrease in proteinuria, hypertension, and glomerulosclerosis compared with the remnant rats not administered these agents. In the final group, remnant rats administered losartan and enalapril followed by aldosterone infusion, degrees of proteinuria, hypertension, and glomerulosclerosis were similar to those of untreated remnant rats. These results further support an independent pathogenetic role for aldosterone as a mediator of progressive renal disease.

It has been reported that continuous angiotensin converting enzyme (ACE) inhibitor therapy does not necessarily produce a maintained decrease in plasma aldosterone levels, which may remain high or increase over time during long-term use (a condition termed “aldosterone escape”). Sato et al. have examined the role of aldosterone escape in 45 patients with type 2 diabetes and early nephropathy treated with an ACE inhibitor for 40 weeks. [Sato A, Hayashi K, Naruse M, Saruta T. Hypertension 41:64 (2003)] With treatment, there was a 40% reduction in average urinary albumin excretion, although urinary albumin excretion in patients with aldosterone escape (18 patients) was significantly higher than that in patients without escape (27 patients). In the 18 patients with escape, spironolactone (25 mg/d) was added to ACE inhibitor treatment in 13 subjects. After a 24-week study period, urinary albumin excretion and left ventricular mass index were significantly reduced without blood pressure change. The authors concluded that aldosterone escape is observed in 40% of patients with type 2 diabetes with early nephropathy despite the use of ACE inhibitors and raised the possibility that aldosterone blockade may represent optimal therapy for patients with early diabetic nephropathy who show aldosterone escape during ACE inhibitor treatment and who no longer show maximal anti-proteinuric effects of ACE inhibition.

### 3.0 Aldosterone Antagonism

3.1 Aldosterone Antagonists. Aldosterone antagonists block aldosterone binding at the mineralocorticoid receptor. Many aldosterone blocking drugs and their effects in humans are known. By way of example, the actions of two aldosterone antagonists that have been approved by the United States Food and Drug Agency are described herein.

For example, the aldosterone antagonist spironolactone binds to the mineralocorticoid receptor and blocks the binding of aldosterone. This steroidal compound has been used for blocking aldosterone-dependent sodium transport in the distal tubule of the kidney in order to reduce edema and to treat essential hypertension and primary hyperaldosteronism [F. Mantero et al., Clin Sci Mol Med, 45 (Suppl 1), 219s-245s (1973)]. Spironolactone is also used commonly in the treatment of other hyperaldosterone-related diseases such as liver cirrhosis and congestive heart failure [F. J. Saunders et al., Aldactone; Spironolactone: A Comprehensive Review, G. D. Searle, New York (1978)]. Its action is relatively nonselective, in that spironolactone binds to recombinant human mineralocorticoid receptors as well as to recombinant human glucocorticoid, progesterone and androgen receptors. Spironolactone has been shown to be pharmacologically effective and well tolerated, to decrease atrial natriuretic peptide concentrations, and reduce the overall risks of death, death due to progressive heart failure, and sudden death from cardiac causes, as well as the risk of hospitalization for cardiac causes. Spironolactone may be used in conjunction with standard doses of an ACE inhibitor, a loop diuretic, and in many cases, digoxin. For example, progressively-increasing doses of spironolactone from 1 mg to 400 mg per day were administered to a spironolactone-intolerant patient to treat cirrhosis-related ascites [P. A. Greenberger et al., N Eng Reg Allergy Proc, 7(4), 343-345 (July-August, 1986)]. Likewise, spironolactone at a dosage ranging from 25 mg to 100 mg daily is used to treat diuretic-induced hypokalemia, when orally-administered potassium supplements or other potassium soaring regimens are considered inappropriate [Physicians' Desk Reference, Medical Economics Company, Inc., Montvale, N.J. (2004)].

Likewise, eplerenone exemplifies another blocker of aldosterone binding at the mineralocorticoid receptor. Its action is selective, in that eplerenone binds to recombinant human mineralocorticoid receptors in preference to binding to recombinant human glucocorticoid, progesterone and androgen receptors. Eplerenone has been shown to produce sustained increases in plasma renin and serum aldosterone, consistent with inhibition of the negative regulatory feedback of aldosterone on renin secretion. The therapeutic benefits associated with administration of eplerenone have been demonstrated in multiple clinical trials. In one such study involving over 6,600 subjects [the Eplerenone Post-Acute Myocardial Infarction Heart Failure Efficacy and Survival Study (EPHESUS)], eplerenone was found to reduce significantly the risk of death attributable to cardiovascular causes and the risk of hospitalization for cardiovascular events. [Pitt B, Remme W, Zannad F, Neaton J, Martinez F, Roniker B, Bittman R, Hurley S, Kleiman J, Gatlin M. New Engl J Med 348 (14), 1309-1321 (2003)] A reduction in the rate of sudden death from cardiac causes was also observed.

3.2 Adverse effects of aldosterone antagonism. After the release of results of the RALES study, there was a rapid increase in the number of prescriptions of spironolactone written for older patients with heart failure who were already being treated with an ACE inhibitor. Unfortunately, there was an equally brisk and striking increase in the number of hospital admissions (starts and subsequent deaths) related to hyperkalemia. Specific to the risk of hyperkalemia associated with aldosterone blockage may be the recent and rapidly increasing use of beta-blockers in patients with heart failure. Beta-blockers not only inhibit renin release, but also act non-selectively to block adrenergic-mediated potassium uptake by peripheral tissues. Co-administration of non-steroidal anti-inflammatory drugs further predisposes a patient to hyperkalemia.

Administration of an aldosterone antagonist to patients with chronic kidney disease and reduced glomerular filtration rate is a major risk factor for hyperkalemia. Serum creatinine is often used as a measure of glomerular filtration rate. In elderly patients, especially women, serum creatinine can underestimate the reduction in glomerular filtration rate (e.g., a creatinine concentration of 2 mg/dl is equal to a glomerular filtration rate of about 26 mL/min/1.73 m sq of body-surface area in 75 year old white woman).

The seriousness of hyperkalemia from use of aldosterone blockers cannot be overemphasized. There have been several reports of serious hyperkalemia following the publication of the RALES study. In one such report, no less than 25 patient episodes of spironolactone-related hyperkalemia that had to be treated in the emergency room were described (Schepkens H, Vanholder R, Billiouw J M, Lameire N. Am J Med 2001; 110:438-441). Four of the 25 patients required cardiovascular resuscitation measures, and 2 of the 25 patients died. Several authors have estimated an incidence of clinically significantly hyperkalemia of about 10% in patients receiving this aldosterone antagonist. Likewise, the label copy that is provided by the manufacturers of each of the various aldosterone antagonists warns in bold type that the principal risk of administration of the aldosterone antagonist is the potentially dangerous development of hyperkalemia. [See, for example, the label copy for INSPRA (eplerenone tablets), NDA 32-437/S-002.] Hyperkalemia can cause serious, sometimes fatal, arrhythmias.

### 4.0 Prevention of Hyperkalemia in Patients Prescribed Aldosterone Antagonists

It is critical that steps be taken to prevent hyperkalemia in patients prescribed aldosterone antagonist. Current approaches for treatment of patients at risk for hyperkalemia caused by inhibitors of the renin-angiotensin-aldosterone system have been reviewed by Palmer. (Palmer, B F. New Engl J Med 2004; 351; 6:585-92). A key recommendation is prescription of diuretics that enhance potassium excretion by the kidney. These diuretics include thiazides and loop diuretics. In patients with a glomerular filtration rate more than 30 mL/min, thiazides can be used. However, when the glomerular filtration rate is less than 30 mL/min, thiazides are not effective, and loop diuretics have to be used.

Potassium is completely filtered at the glomerulus and is reabsorbed in both the proximal nephron and in the loop of Henle. Virtually all of the filtered potassium has been re-absorbed by the time the tubular fluid reaches the end of the loop. Therefore, the potassium that is excreted in the urine is largely derived from the secretory sites (site 4) located in the distal reaches of the nephron. The characteristics of this transport site are such that the more sodium that is delivered to it, the greater the ionic exchange that will occur, and the greater the excretion of potassium will be. Therefore, any diuretic that interferes with sodium re-absorption at a site upstream from site 4 will present additional sodium for exchange, and increased amounts of potassium will appear in the urine.

### 5.0 Metolazone

5.1 Use of metolazone to promote potassium excretion and prevent hyperkalemia. Metolazone is a quinazoline diuretic, with properties similar but not identical to thiazide diuretics. Thiazides such as hydrochlorothiazide inhibit sodium chloride (NaCl) re-absorption from the luminal side of epithelial cells in the distal convoluted tubule of the kidney. In contrast, the primary site of action of metolazone appears to be the early distal convoluted tubule. Metolazone also possesses weak proximal tubular effects, although the mechanism of this action is unclear. Metolazone induces an increase in potassium and titrable acid excretion, due to increased delivery of sodium to the distal tubule.

Metolazone differs from other thiazide diuretics in several significant ways. Metolazone has a tolyl substituent on the quinazoline molecule that provides a prolonged duration of action and increased potency. (Metolazone is about 10 times more potent than hydrochlorothiazide on a weight basis.) Also, while the majority of thiazide diuretics decrease glomerular filtration rate (GFR), thus making them generally ineffective in patients with a GFR of <30 to 50 ml/min, metolazone has been shown to maintain GFR, and efficacy at GFRs as low as 10 ml/min has been demonstrated. Metolazone also impairs sodium-dependent phosphate transport in the proximal tubule, yet another feature that distinguishes metolazone from other thiazide diuretics.

With respect to its effects on renal function, metolazone does not cause any consistent changes in glomerular filtration rate. In addition, the administration of metolazone did not result in any alteration in effective renal plasma flow as estimated by the clearance of p-aminohippurate. Mean values were 489±26 and 460±21 ml/min in the control and peak diuretic periods, respectively. Metolazone reduced the fraction of proximal tubular sodium reabsorbed from 43±3% to 34±4% (p<0.001), while neither the glomerular filtration rate of the whole kidney nor the single nephron GFR was affected by the drug.

Puschett and coworkers have also performed studies comparing the effects of oral and intravenously administered metolazone. Whether metolazone was given intravenously or orally, its effect persisted, whereas the action of chlorothiazide started to decline after about 40 minutes. Puschett took advantage of this effect of metolazone clinically, by giving it every other day, rather than daily, in many of their patients.

The actions in normal healthy male volunteers of metolazone in its original formulation (ZAROXYLYN) have been compared with those of a reformulated product named MYKROX or 25 mg doses of chlorthalidone by Woodworth and his collaborators. Determinations of the mean hourly sodium excretion were obtained for the first 24 hours after drug administration for each of the drug formulations and compared to those produced by 25 mg of chlorthalidone. The findings were as follows:
Sodium excretion following 2.5 mg of ZAROXYLYN was equivalent to that induced by 2.0 mg of MYKROX and both sodium excretion values exceeded natriuresis induced by 25 mg of chlorthalidone.
In the second 24 hours, the natriuresis and diuresis with metolazone in ZAROXYLYN persisted, whereas there was a substantial fall-off with the drug in the MYKROX formulation, regardless of dose.

In a randomized study with a 2×2 factorial design conducted by Channer, et al (Channer K D, McLean K A, Lawson-Matthew O, et al. Br Heart J 1994; 71:146-150), antihypertensive and metabolic effects of metolazone and chlorthalidone were compared in 50 patients with mild-to-moderate essential hypertension. The patients, well-matched for sex and age, were divided into two groups and treated with a starting dose of metolazone (2.5 mg/day) or chlorthalidone for the rest of the three month study period. At the end of treatment, metolazone had induced a significant decrease in both systolic and diastolic blood pressure in the supine and standing positions (p<0.01). However, in the standing position diastolic blood pressure was only slightly reduced (p=0.05) by chlorthalidone, while no significant variations were observed in systolic values. Total serum cholesterol and LDL-cholesterol showed significant increases during chlorthalidone therapy, while no adverse effects on lipid metabolism were observed during metolazone therapy. The difference was statistically significant (p<0.05).

Winchester, et al (Winchester J F, Kellett R J, Boddy K, Boyle P, et al. Clinical Pharmacology and Therapeutics 1980; 28(5) 611-61) performed a double-blind crossover comparison of the effects of 5 mg metolazone and 5 mg bendroflumethiazide on blood pressure and metabolic parameters in 18 non-edematous hypertensive subjects with glomerular filtration rates exceeding 70 ml/min/1.73 m². After a 4-week run-in placebo period, patients received either metolazone or bendroflumethiazide for 6 weeks in crossover fashion with an intervening washout period of 4 weeks. Metolazone induced a more sustained and greater blood pressure response than bendroflumethiazide. Likewise, metolazone induced a greater reduction in total body potassium (TBK) (6.2 gm, 5.5% of TBK) compared to bendroflumethiazide (1.2 gm, 1.1% of TBK, p<0.05). These results suggested that metolazone is a more effective antihypertensive and induces similar but greater metabolic changes than bendroflumethiazide. Changes in plasma potassium and TBK are minor, but they were greater with metolazone.

In a crossover study comparing metolazone, 5 mg once daily, with hydrochlorothiazide (50 mg, twice a day) in patients with essential hypertension, Sambhi, et al. (Sambhi M P, Barrett J D, Eggena P, et al. The Effects of Antihypertensive Therapy Symposium UCLA School of Medicine, Los Angeles, Calif. 1976) found that metolazone was significantly more effective than hydrochlorothiazide as a hypotensive agent. Decrease in blood potassium levels (hypokalemia) was more marked with metolazone.

5.2 Use of metolazone in patients with renal insufficiency/failure. Several investigators have reported the beneficial use of metolazone in small studies of patients suffering from renal failure and/or nephrotic syndrome with severely reduced glomerular filtration rates (GFR). Several of these studies are summarized below:
Craswell et al., (Craswell P W, Ezzat E, Kopstein J, et al. Nephron 1973; 12:63-73) administered intravenous metolazone to 8 hospitalized patients with impaired renal function, to study the acute effects of the drug on renal function. Intravenous metolazone in a dose of 5 mg produced significant increases in both urine flow and urinary sodium excretion (relative to baseline). Inulin clearance increased in 4 patients (average of 19.2 ml/min/1.73 m²). Subsequently, 12 outpatients were treated with oral metolazone to determine efficacy and side effects with long-term administration. All 12 patients lost weight; 6 of 7 patients with edema on presentation achieved successful removal of all traces of edema fluid. Seven of the 12 patients were hypertensive (diastolic blood pressure >90 mmHg) on entering the trial; all had lower pressures at the end of the study (duration ranged 3 to 20 weeks) with maintenance doses of 2.5 mg (n=2), 5 mg (n=3), 10 mg (n=1), and 15 mg (n=1). Creatinine clearances were determined in 8 of 12 patients; small, nonsignificant increases in creatinine clearance were observed.
Dargie et al., (Dargie H J, et al. Brit Med Jour 1972; 4:196-198) reported achieving satisfactory diureses in sixteen acute studies performed on 14 patients with non-edematous stable chronic renal failure with creatinine clearances ranging from 1.2 to 12 ml/min. The study participants were treated with oral doses of metolazone ranging from 20 to 150 mg. Peak diuretic effect occurred an average of 6 hours after administration, during which a small but statistically significant increase in free water clearance was found. Although the pre-treatment mean GFR was 4.2 ml/min, the mean urine flow rate achieved post-treatment was 2.36 ml/min, an increase of 51%. Mean sodium excretion increased 113%, and potassium excretion increased 33%. The authors reported no major side effects to metolazone treatment, and the drug was well tolerated. These results show that, even in patients with advanced chronic renal failure (CRF), metolazone can induce significant sodium and potassium wasting.
Paton and Kane (Patron R R, Kane R E. The Journal of Clinical Pharmacology 1997; 17(4):243-251) conducted a study to determine the effect of long-term diuretic therapy with metolazone in patients with chronic renal failure and nephrotic syndrome. Twenty nonhospitalized patients having pitting edema due to renal disease were given metolazone for up to 44 months. The study participants were treated with doses of metolazone ranging from 2.5 mg to 20 mg given once daily. Metolazone produced a significant natriuretic and diuretic effect in patients with both CRF and nephrotic syndrome. Additionally, study participants' benefits included loss of edema, improved control of blood pressure (average decrease of 172/97 to 158/88 mmHg and 147/97 to 137/88 mmHg in patients with CRF and nephrotic syndrome, respectively), and improved control of fluid retention.
Bennett and Porter (Bennett W M, Porter G A. J Clin Pharm 1973; 13(8,9):357-364) conducted a clinical trial of metolazone in 20 outpatients (11/20 patients were on concomitant steroid) with edema due to nephrotic syndrome and/or chronic renal disease over 3 months. Three patients had nephrotic syndrome with proteinuria >3 g/24 hours, serum albumin <3 gram %, edema, and hypercholesterolemia. The remainder had chronic depression of GFR with creatinine clearance <50 mL/min from a variety of causes. Metolazone was administered orally in doses starting at 10-15 mg daily, and was increased in weekly 5 mg increments until edema was decreased, or until a maximum dose of 25 mg was reached. There were no adverse effects on renal function. Eleven of the 13 patients with baseline diastolic blood pressure >90 mmHg achieved significant lowering of blood pressure with metolazone (mean decrease was 12.2 mmHg). Twelve of the 20 patients experienced a decrease in serum potassium, and 5 fell below 3.5 mEq/L, requiring potassium (KCl) supplementation.

The combination drug ALDACTAZIDE provides co-administration of the aldosterone antagonist spironolactone with the thiazide diuretic hydrochlorothiazide. The instant invention discloses the combination of an aldosterone antagonist agent with a metolazone-related compound for use in the treatment of hypertension, congestive heart failure, and chronic kidney disease, especially of the proteinuric variety.

### SUMMARY OF THE INVENTION

The present invention is a pharmaceutical composition of an aldosterone antagonist agent and a metolazone-related compound for use in the treatment of hypertension, congestive heart failure, and chronic kidney disease, especially of the proteinuric variety. A method of treating hypertension, congestive heart failure, and chronic kidney disease in a warm-blooded animal with a therapeutically effective dose amount of a pharmaceutical composition of an aldosterone antagonist agent and a metolazone-related compound is disclosed. Included within the scope of the term “pharmaceutical composition” are a fixed dose combination and a concomitant therapy of a dose of an aldosterone antagonist agent and a dose of the diuretic, together with other medications for the treatment of heart disease.

### DETAILED DESCRIPTION OF THE INVENTION

The present invention relates to a pharmaceutical composition of an aldosterone antagonist agent and a metolazone-related compound for the treatment of hypertension, congestive heart failure, and chronic kidney disease, especially of the proteinuric variety. The present invention also relates to the method of treating hypertension, congestive heart failure, and chronic kidney disease in a warm-blooded animal by coadministering to the animal in need of such treatment a therapeutically effective amount of a pharmaceutical composition of an aldosterone antagonist agent and metolazone or their pharmaceutically acceptable salts or prodrugs in a pharmaceutically acceptable carrier, with either concomitant therapy or a fixed combination of the aldosterone antagonist agent and metolazone.

The phrase “combination therapy” (or “co-therapy” or “concomitant therapy”), in defining use of an aldosterone antagonist agent and a metolazone-related compound, is intended to embrace administration of each agent in a sequential manner in a regimen that will provide beneficial effects of the drug combination, and is intended as well to embrace co-administration of these agents in a substantially simultaneous manner, such as by oral ingestion of a single dosage form having a fixed ratio of these active agents or ingestion of multiple, separate capsules for each agent. A fixed dose combination suitable for oral ingestion would be in the form of a tablet, capsule, elixir or pharmaceutically acceptable oral dosage form and comprises between 1 mg to about 500 mg of the aldosterone antagonist agent and between 1 mg to about 50 mg of a metolazone-related compound, and a pharmaceutical carrier. A preferred embodiment is a pharmaceutical composition consisting of about 25 or 50 mg of the aldosterone antagonist agent, and 2.5 or 5 mg of metolazone, and a pharmaceutical carrier. “Combination therapy” also includes simultaneous or sequential administration by intravenous, intramuscular, or other parenteral routes into the body, including direct absorption through mucous membrane tissues, as found in the sinus passages. Sequential administration also includes drug combination where the individual agents may be administered at different times and/or by different routes but which act in combination to provide a beneficial effect.

The phrase “therapeutically effective” is intended to qualify the amount of each agent for use in the combination therapy which will achieve the goal of improvement in cardiac sufficiency by reducing or preventing, for example, the progression of congestive heart failure, while avoiding adverse side effects typically associated with each agent.

A preferred combination therapy will consist essentially of two active agents, namely an aldosterone antagonist agent and a metolazone-related compound. The active agents will be used in combination in a weight ratio range from about 1.0-to-one to about 500-to-one of the aldosterone antagonist agent to the metolazone-related compound. A preferred range of these two agents (aldosterone antagonist agent-to-metolazone-related compound) would be from about four-to-one to about 40-to-one, while a more preferred range would be from about ten-to-one to about twenty-to-one, depending ultimately on the selection of the aldosterone antagonist agent.

The phrase “metolazone-related compound” includes within the scope of this invention metolazone, a salt form of metolazone, and a prodrug form of metolazone comprising a compound that is converted by chemical or biological action in the body of a warm-blooded animal to metolazone. Metolazone has the molecular formula C₁₆H₁₆ClN₃O₃S, the chemical name 7-chloro-1,2,3,4-tetrahydro-2-methyl-3-(2-methylphenyl)-4-oxo-6-quinazolinesulfonamide, a mole-cular weight of 365.83, and the Chemical Abstracts Service (CAS) Registry Number of 17560-51-9. Metolazone is described in U.S. Pat. No. 3,360,518, and has a melting point of 256° C., an octanol:water partition coefficient of 1.84, and a solubility in water of 60.3 mg/L at 25° C. Metolazone is a potent, long-acting diuretic useful in chronic renal disease. Metolazone is currently being marketed in the United States under the tradename ZAROXOLYN (CellTech) or as therapeutic equivalents labeled generically as “Metolazone” (Eon, Mylan, Teva, Roxane, Watson) in a 2.5, 5, or 10 mg dose.

The phrase “aldosterone antagonist agent” comprises an agent or compound, or a combination of two or more of such agents or compounds, which counteracts the effects of aldosterone. Such agents and compounds, such as mespirenone, may antagonize the action of aldosterone through pre-receptor mechanisms. Other agents and compounds, such as spironolactone and eplerenone, fall generally within a class known as aldosterone receptor antagonists and bind to aldosterone receptors such as typically are found in renal tubules, and prevent natural ligand activation of post-receptor events. A family of aldosterone antagonists having spirolactone-type formulae and methods to make compounds in this family are described in U.S. Pat. No. 4,129,564 to Wiechart et al. A second family of spirolactone-type compounds and methods to make the compounds in this second family are described in U.S. Pat. No. 4,789,668 to Nickisch et al. A third family of spirolactone compounds and methods to make the compounds in this third family are described in U.S. Pat. No. 3,257,390 to Patchett. Of particular interest is the compound spironolactone, which is described in U.S. Pat. No. 3,013,012 to Cella et al., and epoxy steroids, including in particular, the compound eplerenone, which are described in U.S. Pat. No. 4,559,332 to Grob et al. and in WO97/21720 to Ng et al. and WO98/25948 to Ng et al.

Spironolactone has the molecular formula C₂₄H₃₂O₄S, the chemical name 17-hydroxy-7α-mercapto-3-oxo-17α-preg-4-ene-21-carboxylic acid, γ-lactone acetate, a molecular weight of 416.58, and the CAS Registry Number of 52-01-7. Spironolactone is currently being marketed in the United States under the tradename ALDACTONE in a dose of 25, 50, or 100 mg of the active ingredient spironolactone.

Also within the scope of this invention is a prodrug form of spironolactone comprising a compound that is converted by chemical or biological action in the body of a warm-blooded animal to spironolactone or a therapeutically active metabolite of spironolactone.

Eplerenone has the molecular formula C₂₄H₃₀O₆, the chemical name (7α,11α,17α)-9,11-epoxy-17-hydroxy-3-oxo-pregn-4-ene-7,21-dicarboxylic acid γ-lactone methyl ester, a molecular weight of 414.49, and the CAS Registry Number of 107724-20-9. The octanol:water partition coefficient is 7.1 at pH 7.0. Eplerenone is currently being marketed in the United States under the tradename INSPRA (Pfizer) in a 25, 50 or 100 mg dose of the active ingredient eplerenone.

Also within the scope of this invention is a prodrug form of eplerenone comprising a compound that is converted by chemical or biological action in the body of a warm-blooded animal to eplerenone or a therapeutically active metabolite of eplerenone.

Included within the scope of this invention is a method of treating hypertension, congestive heart failure, and chronic kidney disease in a warm-blooded animal using pharmaceutical compositions comprising an aldosterone antagonist and metolazone and a suitable pharmaceutical carrier.

For the purpose of this disclosure, a warm-blooded animal is a member of the animal kingdom which includes but is not limited to mammals and birds. The most preferred mammal of this invention is human.

Surprisingly, the inventor has discovered that the combination of an aldosterone antagonist agent with a metolazone-related compound provides unexpected and synergistic advantages to the patient. Combinations of the present invention provide dosages of individual drugs in the combinations disclosed in Table 1, where preferred combinations of dosages comprise a combination of the present invention consisting of the specific dosage of aldosterone antagonist agent in combination with each of the metolazone dosages for that combination that are shown in brackets. Most preferred combinations of dosages comprise a combination of the present invention consisting of the specific dosage of aldosterone antagonist agent, wherein the agent is spironolactone or eplerenone, in combination with each of the metolazone dosages for that combination that are shown in brackets.

TABLE 1
Dosages of individual drugs in combinations of the present invention

| Combination of the | Aldosterone Antagonist | |
|---|---|---|
| Present Invention | Agent (mg) | Metolazone (mg) |
| 1 | 12.5 | 1-5 [1, 2.5] |
| 2 | 25 | 1-20 [2.5, 5, 10] |
| 3 | 37.5 | 1-20 [2.5, 5, 10] |
| 4 | 50 | 2.5-20 [2.5, 5, 10, 20] |
| 5 | 100 | 2.5-20 [5, 10, 20] |
| 6 | 150 | 2.5-20 [5, 10, 20] |
| 7 | 200 | 2.5-20 [5, 10, 20] |
| 8 | 400 | 2.5-50 [5, 10, 20, 50] |

The novel drug combinations of this invention have the following desirable properties. The combination therapy comprises administering an aldosterone antagonist agent and a metolazone-related compound at doses that in combination result in one or more of the following: (1) a statistically significant reduction in the death rate as compared to said combination therapy without metolazone; (2) a statistically significant reduction in the number of non-fatal hospitalizations as compared to said combination therapy without metolazone; (3) a statistically significant reduction in the number of sudden deaths from cardiac causes, as well as the risk of hospitalization for cardiac causes. There are also fewer side effects with the combination, since the drugs act synergistically in reducing the overall risks of death, death due to progressive heart failure, and sudden death from cardiac causes, as well as the risk of hospitalization for cardiac causes. The combination therapy of the present invention has a low toxicity and provides significantly improved safety to the patient in that the metolazone-related compound prevents the hyperkalemia associated with administration of the aldosterone antagonist agent, thereby improving the safety profile of the aldosterone antagonist agent, and the aldosterone antagonist agent prevents the hypokalemia associated with administration of the metolazone-related compound, improving thereby the safety profile of the metolazone-related compound. Furthermore, impairment of glomerular filtration in some patients by thiazide diuretics further increases the risk for hyperkalemia, when thiazides are used in combination with aldosterone antagonists. In contrast, metolazone does not impair glomerular filtration and therefore is superior to thiazides when combined with aldosterone antagonists. Lastly and most importantly, the combination is active upon oral administration.

In addition, co-administration of an aldosterone antagonist agent with a metolazone-related compound as disclosed in the present invention provides the advantages of significantly improved patient compliance with prescribed dose regimens. Since the incidence of cardiovascular disease is highest among human subjects of age 65 or older, maintenance of compliance with a prescribed dose regimen is particularly important in optimizing the effectiveness of the prescribed therapy. A major risk of prescribing metolazone and an aldosterone antagonist separately and not as a combination tablet/capsule is the clinical reality that, either inadvertently or deliberately, a significant number of patients may take only one and not both of the separate medications. If the patient takes metolazone without the aldosterone antagonist, the patient does not derive the cardio-protective or renal-protective effect. On the other hand, if the patient takes an aldosterone antagonist without metolazone, absent its kaliuretic action, the patient is pre-disposed to hyperkalemia. Therefore, the combination therapy of the present invention comprising an aldosterone antagonist agent with metolazone in one tablet/capsule eliminates the possibility of a patient taking one medication without the other. Furthermore, co-administration of the combination of drugs of the present invention simplifies the dosage regime in that it reduces the number of tablets, capsules, or oral doses that must be ingested by the patient at each dosage interval. A reduction in the number of tablets that must be ingested prevents errors in dosage associated with, by way of example, ingestion of too few or too many tablets, the wrong combination of tablets, and the like. Likewise, a reduction in number of tablets, capsules, or other oral dosage forms that must be ingested by the patient at each dosage interval also reduces the incidence of dysphagia, problems with swallowing, regurgitation, and the like, thereby preventing the development of resistance to a therapeutic dosage regimen and improving patient compliance with the dosage regimen.

The dosage administered will be dependent on the age, health and weight of the recipient, the extent of disease, the presence of co-morbidities, kind of concurrent treatment, if any, frequency of treatment and the nature of the effect desired. Usually, a daily dosage of the active ingredient compounds will be from about 1 mg to about 500 mg of an aldosterone antagonist agent and from about 0.5 mg to about 50 mg of a metolazone-related compound.

The particular dose for each specific patient depends on diverse factors, including, for example, the age, the body weight, the general state of health, the sex, and the diet of the patient; on the time and route of administration; on the rate of excretion; on the combination of medications being taken by the patient; and on the severity of the particular disorder for which therapy is being given.

### Dosage Forms

The pharmaceutical compositions of this invention can be administered by any means that effects contact of the active ingredients with the site of action in the body of a warm-blooded animal. For example, the means can be oral, transdermal, by inhalation, or parenteral (i.e., subcutaneous, intravenous, intramuscular or intraperitoneal). Alternatively or concurrently, the means of administration can be by more than one route (e.g., oral and parenteral). A most preferred means of administration is by the oral route (i.e., ingestion).

The active ingredients can be administered by the oral route in solid dosage forms, such as tablets, capsules, and powders, or in liquid dosage forms, such as elixirs, syrups, and suspensions. The pharmaceutical compositions of this invention also can be administered parenterally, in sterile liquid dosage forms. The pharmaceutical composition is preferably made in the form of a dosage unit containing a particular amount of each active ingredient.

In general, the pharmaceutical compositions of this invention can be prepared by conventional techniques, as are described in Remington's Pharmaceutical Sciences, a standard reference in this field [Gennaro A R, Ed. Remington: The Science and Practice of Pharmacy. 20^{th} Edition. Baltimore: Lippincott, Williams & Williams, 2000]. For therapeutic purposes, the active components of this combination therapy invention are ordinarily combined with one or more adjuvants appropriate to the indicated route of administration. If administered per os, the components may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tabletted or encapsulated for convenient administration. Such capsules or tablets may contain a controlled-release formulation as may be provided in a dispersion of active compound in hydroxypropyl methylcellulose. Solid dosage forms can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract. Both the solid and liquid oral dosage forms can contain coloring and flavoring to increase patient acceptance.

For example, an oral dosage may be prepared by screening and then mixing together the following list of ingredients in the amounts indicated (Table 2). The dosage may then be placed in a hard gelatin capsule.

TABLE 2
Formulation for an oral dosage of a combination
of the present invention

| Ingredients | Amounts |
|---|---|
| Spironolactone | 50.0 mg |
| Metolazone | 2.5 mg |
| Magnesium stearate | 10 mg |
| Lactose | 100 mg |

Alternatively and by way of example, an oral dosage may be prepared by mixing together and granulating with a 10% gelatin solution. The wet granules are screened, dried, mixed with starch, talc and stearic acid, screened and compressed into a tablet having a composition as described in Table 3.

TABLE 3
Formulation for an oral dosage of a composition of the present invention

| Ingredients | Amounts |
|---|---|
| Eplerenone | 50.0 mg |
| Metolazone | 5.0 mg |
| Calcium sulfate dihydrate | 100 mg |
| Sucrose | 15 mg |
| Starch | 8 mg |
| Talc | 4 mg |
| Stearic acid | 2 mg |

Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The components may be dissolved in water, polyethylene glycol, propylene glycol, ethanol, corn oil, cottonseed oil, peanut oil, sesame oil, benzyl alcohol, sodium chloride, and/or various buffers. The indicated formulations can contain compatible auxiliaries and excipients, such as anti-oxidants, preservatives, stabilizing agents, emulsifiers, salts for influencing the osmotic pressure, and/or buffer substances.

Other adjuvants and modes of administration are well and widely known in the pharmaceutical art.

Pharmaceutical compositions for use in the treatment methods of the invention may be administered in oral form or by intravenous administration. Oral administration of the combination therapy is preferred. Dosing for oral administration may be with a regimen calling for single daily dose, or for a single dose every other day, or for multiple, spaced doses throughout the day. The active agents which make up the combination therapy may be administered simultaneously, either in a combined dosage form or in separate dosage forms intended for substantially simultaneous oral administration. The active agents which make up the combination therapy may also be administered sequentially, with either active component being administered by a regimen calling for two-step ingestion. Thus, a regimen may call for sequential administration of the active agents with spaced-apart ingestion of the separate, active agents. The time period between the multiple ingestion steps may range from a few minutes to several hours, depending upon the properties of each active agent such a potency, solubility, bioavailability, plasma half-life and kinetic profile of the agent, as well as depending upon the age and condition of the patient. The active agents of the combined therapy whether administered simultaneously, substantially simultaneously, or sequentially, may involve a regimen calling for administration of one active agent by oral route and the other active agent by intravenous route. Whether the active agents of the combined therapy are administered by oral or intravenous route, separately or together, each such active agent will be contained in a suitable pharmaceutical formulation of pharmaceutically-acceptable excipients, diluents or other formulation components. Examples of suitable pharmaceutically-acceptable formulations containing the active components for oral administration are given below. Even though such formulations list both active agents together in the same recipe, it is appropriate for such recipe to be utilized for a formulation containing one of the active components.

Without further elaboration, it is believed that one skilled in the art can, using the preceding description, utilize the present invention to its fullest extent. The following specific embodiments are, therefore, to be construed as merely illustrative, and not limitative of the remainder of the disclosure in any way whatsoever.

The following examples present hypothetically useful therapeutic applications of representative pharmaceutical compositions of the present invention and their anticipated outcomes in treating hypertension, heart failure and chronic kidney disease in subjects requiring such treatment. The examples are representative of the scope of the invention, and as such are not to be considered or construed as limiting the invention recited in the appended claims.

### EXAMPLE 1

### Aldactone 25 mg+Metolazone 1 mg Combination as an Initial Anti-Hypertensive Therapy in a Patient with Hypertension

A 23 year old black male presents to the family physician with headaches. The patient's blood pressure is found to be 160/100 mmHg, a value which is again confirmed on a repeat visit after 1 week. The patient is started on 12.5 mg hydrochlorothiazide daily. After 1 month, the patient's blood pressure is 150/90 and his serum potassium is 3.0 mEq/L (hypokalemic). Hydrochlorothiazide is replaced by a combination of 1 mg metolazone and 25 mg spironolactone every morning. Over next 2 months, the patient's blood pressure decreases to 135/80 mm of Hg. His serum potassium remains stable in the 4.0-4.2 mEq/L range.

### EXAMPLE 2

### Aldactone 25 mg+Metolazone 2 mg Combination as an Addition to the Current Anti-Hypertensive Regimen of a Patient with Hypertension

A 65 year old male with hypertension is seen by an internist in the outpatient clinic. The patient's blood pressure is 170/85 (170 mm of Hg systolic and 85 mm of Hg diastolic) despite ingestion of the maximum dose of ACE-1. Serum creatinine is 1.1 mg/dl. Hydrochlorothiazide is added in a dose of 25 mg every morning. After 2 months, the patient's blood pressure is 150/80 and serum potassium is 3.5 mEq/L (hypokalemic). Hydrochlorothiazide is replaced by a combination of 2 mg metolazone and 25 mg spironolactone every morning. Over the next 2 months, the patient's blood pressure decreases to 135/80 mm of Hg. His serum potassium remains stable in the 4.4-4.8 mEq/L range.

### EXAMPLE 3

### Aldactone 25 mg+Metolazone 2 mg Combination in a Patient with Chronic Kidney Disease and Congestive Heart Failure

A 78 year old female patient is referred to a cardiologist with progressive dyspnea, edema, orthopnea and paroxysmal nocturnal dyspnea over the previous three months, despite institution of a diuretic, furosemide, 40 mg daily and an angiotensin converting enzyme inhibitor (ACE-I), ramipril, 10 mg daily. Physical examination reveals a frail patient weighing 42 kilograms with congestive heart failure. An echocardiogram reveals global hypokinesis with a left ventricular ejection fraction of 30%. The patient's serum potassium is 5.0 mEq/L and serum creatinine is 1.6 mg/dl.

Glomerular filtration rate is not calculated, and the cardiologist adds 25 mg spironolactone as a treatment that has been shown to reduce mortality in patients with congestive heart failure. The patient presents to the emergency room two weeks later with weakness. Serum potassium is 6.3 mEq/L. Hyperkalemia is urgently treated, ramipril and spironolactone are discontinued, and the patient is discharged. Post-treatment review leads to the conclusion that the patient's plasma creatinine of 1.6 mg/dl had led the cardiologist to overestimate renal function and prescribe spironolactone, while the calculated glomerular filtration rate is only 22 ml/min. On subsequent visits to the cardiology clinic, patient is started back on 10 mg ramipril in combination with a long acting diuretic, metolazone 2.5 mg. Serum creatinine and potassium have decreased to 1.4 mg/dl and 4.6 mEq/L. On the next clinic visit, 25 mg spironolactone is added. Serum potassium and creatinine are serially followed every week for the next 3 weeks and remain unchanged. Patient is switched to a combination of 2 mg metolazone and 25 mg spironolactone to ensure that metolazone does not get discontinued inadvertently if the patient continues to take spironolactone alone, and thereby lead to hyperkalemia,

### EXAMPLE 4

### Spironolactone 25 mg+Metolazone 2 mg Combination in a Patient with Proteinuric Chronic Kidney Disease, Stage 2

A 35 year old insulin-dependent diabetic subject presents to the nephrologists with a glomerular filtration rate of 65 ml/min and 2 grams proteinuria per day despite ingestion of maximum doses of ACE-I. To further reduce proteinuria and slow the progression of renal disease, 25 mg spironolactone daily is added. After 3 months, the patient's proteinuria decreases to 1 gm per day. However, serum potassium rises to 6.5 mEq/L, necessitating the discontinuation of spironolactone. Proteinuria increases to 2.2 gm per day. Subsequently, a combination of 2 mg metolazone and 25 mg spironolactone is started. Over the next 4 months, proteinuria decreases to 1 gm per day. Serum potassium remains stable in the 4.8-5.1 mEq/L range.

### EXAMPLE 5

### Aldactone 25 mg+Metolazone 5 mg Combination, in a Patient with Proteinuric Chronic Kidney Disease, Stage 4

A 65 year old non-insulin dependent diabetic subject presents to the nephrologists with a glomerular filtration rate of 18 ml/min and 1.8 grams proteinuria per day despite ingestion of maximum doses of ACE-I. Edema is controlled by 40 mg furosemide every morning. To further reduce proteinuria and slow the progression of renal disease, 25 mg spironolactone daily is added. After 3 months, the patient's proteinuria decreases to 1 gm per day. However, serum potassium rises to 6.3 mEq/L, necessitating the discontinuation of spironolactone and ACE-I. The patient's proteinuria increases to 2.2 gm per day. Subsequently, ACE-I treatment is restarted, and furosemide is replaced by a combination of 2 mg metolazone and 25 mg spironolactone every morning. Over the next 4 months, the patient's proteinuria decreases to 1.1 gm per day. The patient's serum potassium remains stable in the 4.8-5.1 mEq/L range.

### EXAMPLE 6

### Aldactone 50 mg+Metolazone 2.5 mg Combination for Treatment of Ascites in a Patient with Alcoholic Cirrhosis

A 50 year old female is admitted to the hospital with decompensated cirrhosis and massive ascites. Patient is treated over a period of one week with escalating doses of spironolactone and furosemide. Ascites shows only minimal improvement with 100 mg spironolactone and 80 mg furosemide daily in two divided doses. After 1 week, spironolactone is replaced by 2 tablets of a combination of Aldactone 50 mg+Metolazone 2.5 mg, leading to a significant resolution of ascites over the next week.

### EXAMPLE 7

### Epleronone 50 mg+Metolazone 5 mg Combination in a Patient with Chronic Kidney Disease and Congestive Heart Failure

A 45 year old male patient with past history of hypertension and anterior myocardial infarction is referred to a cardiologist with progressive dyspnea, edema, orthopnea and paroxysmal nocturnal dyspnea over the previous six months, despite administration of a diuretic, furosemide, 40 mg daily, and an angiotensin II receptor blocker (ARB), losartan, 100 mg daily. Physical examination reveals a patient weighing 68 kilograms with congestive heart failure. An echocardiogram reveals global hypokinesis with a left ventricular ejection fraction of 25%. The patient's serum potassium is 4.9 mEq/L and serum creatinine is 1.9 mg/dl. The cardiologist adds 50 mg epleronone as a treatment that has been shown to reduce mortality in patients with congestive heart failure. The patient presents to the emergency room two weeks later with weakness. Serum potassium is 6.4 mEq/L. Hyperkalemia is urgently treated, ARB and epleronone are discontinued, and the patient is discharged. Post-treatment review suggests that hyperkalemia was a consequence of administration of an aldosterone antagonist and ARB to a patient with impaired renal function. On subsequent visits to the cardiology clinic, patient is started back on losartan 100 mg in combination with a long acting diuretic, metolazone 5.0 mg. Serum creatinine and potassium have decreased to 4.5 mEq/L. On the next clinic visit, 25 mg epleronone is added. Serum potassium and creatinine are serially followed every week for the next 3 weeks and remain unchanged. Patient is switched to a combination therapy of this invention of 5 mg metolazone and 50 mg epleronone to ensure that metolazone does not get discontinued inadvertently if the patient continues to take the aldosterone receptor blocker alone, and thereby lead to hyperkalemia.

### EXAMPLE 8

### A Combination Therapy of the Present Invention Comprising Epleronone 50 mg+Metolazone 5 mg in a Patient with Diabetic Nephropathy and Congestive Heart Failure

A 40 year old male patient with diabetic nephropathy, nephrotic syndrome, GFR of 25 ml per minute, coronary artery disease and CHF is currently maintained edema-free on 5 mg metolazone and ACE-I (an ACE inhibitor). To slow the progression of renal and cardiac disease, the nephrologist decides to add epleronone to the dosage regimen. He explains to the patient that a new “water pill” (i.e., epleronone) is being added. The patient misunderstands the instructions and believes that the current water pill (i.e., metolazone) is being replaced by the new pill. The patient stops taking metolazone and continues to take epleronone and ACE-I. After two weeks, the patient presents to the emergency room with severe shortness of breath secondary to pulmonary edema. The patient's plasma potassium level is 6.4 mEq/L. It is concluded that fluid overload and hyperkalemia are the consequences of discontinuing metolazone. In order to prevent further mistakes, the patient is prescribed a combination therapy of the present invention comprising a pill containing 5 mg metolazone and 50 mg epleronone. The patient's condition improves.

### EXAMPLE 9

### A Combination Therapy of the Present Invention Comprising Spironolactone 50 mg+Metolazone 2.5 mg in a Patient with Diabetic Nephropathy

A 45 year old male patient with diabetic nephropathy has daily proteinuria of 3 gm despite ingestion of the maximum dose of an ACE-I and a GFR of 30 ml/min. The patient is prescribed 25 mg spironolactone to further reduce proteinuria. Daily proteinuria decreases to 2.2 gm and further decreases to 1.5 gm when the dose of spironolactone is increased to 50 mg daily. After 6 months, the patient develops worsening congestive heart failure, and the cardiologist decides to add 2.5 mg metolazone daily. He explains to the patient that a new “water pill” (i.e., metolazone) is being added. The patient misunderstands the instructions and believes that the current water pill (i.e., spironolactone) is being replaced by the new pill (i.e., metolazone). The patient stops taking spironolactone and continues to take metolazone and ACE-I. Four months later the patient presents to the nephrologists with worsening edema. The patient's plasma potassium level is 2.9 mEq/L, and proteinuria has increased to 3.2 gm per day. It is concluded that the increase in proteinuria and hypokalemia are the consequences of discontinuing spironolactone. In order to prevent further mistakes, the patient is prescribed a combination therapy of the present invention comprising a pill containing 2.5 mg metolazone and 50 mg spironolactone. The patient's condition improves.

All mentioned references are incorporated by reference as if here written. When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

1-32. (canceled)

33. A combination therapy for a patient in need of treatment for congestive heart failure, hypertension, or chronic kidney disease comprising:
co-administering a therapeutically effective dose of metolazone and eplerenone.

34. The combination therapy of claim 33, wherein the daily dose of metolazone is in the range from about 1 mg to about 50 mg and the daily dose of eplerenone is in the range of about 1 mg to about 400 mg.

35. The combination therapy of claim 33, wherein the metolazone in administered in a pharmaceutically acceptable salt, ester or prodrug.

36. The combination therapy of claim 33, wherein the chronic kidney disease is diabetic nephropathy.

37. A combination therapy for a subject in need of treatment for congestive heart failure and proteinuric kidney disease comprising:
co-administering metolazone and an aldosterone agonist agent in a therapeutically effective dose.

38. The combination therapy of claim 37 wherein the daily dose of metalozone is in the range from about 1 mg to about 50 mg and the daily dose of the aldosterone agonist is in the range of about 1 mg to about 400 mg.

39. The combination therapy of claim 37 wherein the metolazone in administered in a pharmaceutically acceptable salt, ester or prodrug.

40. A combination therapy for a subject in need of treatment for heart failure and chronic kidney disease comprising:
a first amount of metolazone and a second amount of an aldosterone agonist agent, wherein the first and second amount together comprise a therapeutically effective amount to treat heart failure and chronic kidney disease, wherein the aldosterone agonist agent is eplerenone or spironolactone.

41. The combination therapy of claim 40, wherein said aldosterone agonist agent is mespirenone.

42. A composition comprising:
a first amount of an aldosterone antagonist agent;
a second amount of a metolazone; and
a third amount of a pharmaceutically acceptable carrier.

43. A composition comprising:
a first amount of eplerenone;
a second amount of a metolazone; and
a third amount of a pharmaceutically acceptable carrier.
